(19) United States
(12) Patent Application Publication
(10) Pub. No.: US 2015/0133323 A1
(43) Pub. Date: May 14, 2015

(54) METHOD FOR CHARACTERIZING CIRCULATING TUMOR CELLS, AND USE THEREOF IN DIAGNOSIS

(71) Applicant: INSTITUT GUSTAVE ROUSSY, Villejuif Cedex (FR)

(72) Inventors: Amélie Barthelemy, Vitry Sur Seine (FR); Francoise Farace, Montrouge (FR); Marianne Oulhen, Paris (FR); Jean-Charles Soria, Igny (FR); Alexander Valent, Paris (FR); Philippe Vielh, Sceaux (FR); Benjamin Besse, Paris (FR); Emma Pailler, Paris (FR)

(73) Assignees: UNIVERSITE PARIS SUD XI, Orsay cedex (FR); INSTITUT GUSTAVE ROUSSY, Villejuif Cedex (FR)

(21) Appl. No.: 14/358,836

(22) PCT Filed: Nov. 19, 2012
(86) PCT No.: PCT/FR2012/000471

§ 371 Date: May 16, 2014

(30) Foreign Application Priority Data
Nov. 17, 2011 (FR) PCT/FR2011/052689

(51) Int. Cl. C12Q 1/68; G01N 33/574

(52) U.S. Cl. CPC C12Q 1/6888; G01N 33/57496; G01N 33/57492; G01N 33/57423; G01N 33/57434; G01N 33/57415; C12Q 2600/158; G01N 2333/4742; G01N 2333/70589; G01N 2333/91205
USPC 506/9; 435/6.11; 435/6.12; 435/29; 435/7.1; 506/7; 435/7.23

(57) ABSTRACT

Disclosed is a method for characterization, in a biological sample, of circulating tumour cells (CTCs) bearing at least one marker characteristic of the tumorous nature of the cell, the marker being selected from the groups constituted by: the oncogenic proteins characteristic of the CTCs, and the tumour markers. Also disclosed is the use of this method for deciding on the implementation of a treatment for a cancer patient.

The present invention relates to a new method of characterizing circulating tumour cells (CTCs) as well as the uses of this method for deciding on the implementation of a treatment, diagnosing the state of progression of a cancer and obtaining a prognosis of the evolution of the disease in a patient.

Circulating tumour cells play a crucial role in the process of metastasis, and analysis of them can supply important information for a patient's prognosis. Furthermore, they constitute tumorous material that is easily accessible by non-invasive methods, representative of the metastatic disease in real time. In the context of the development of personalized medicine for cancer, detection of the presence of molecular target markers of an antineoplastic treatment in CTCs can allow selection of patients who are likely to benefit from this treatment or even evaluation of the response or resistance to this same treatment at the individual level. Selection of patients likely to benefit from a targeted antineoplastic treatment is currently based on searching for molecular markers in biopsies of metastases. The present method makes it possible to perform this search on CTCs, supplementing or even replacing analysis of the tumour biopsy.

The current difficulties in detecting CTCs are connected with the fact that they are present in small numbers in the blood. In the context of targeted antineoplastic treatments, diagnosis of the presence of a molecular marker in CTCs must therefore be carried out on very few cells. To establish this diagnosis reliably and with certainty, different analyses must therefore be carried out in parallel on the cells in order to show on the one hand the tumorous nature of the CTCs on the basis of the phenotype (tumour markers), the morphology and a molecular abnormality implicated in oncogenesis. (for example: rearrangement of the ALK gene, amplification of the HER2 gene).

Lecharpentier et al. (2011) describe a method for detecting CTCs in a blood sample, based on the co-expression by said cells of specific markers of epithelial cells and of specific markers of mesenchymal cells. This method employs the combined use of three fluorescent markers and requires an additional morpho-cytological analysis, carried out by an experienced cytopathologist.

The method according to the present invention offers an alternative solution for detecting CTCs that does not require the presence of an accredited cytopathologist, as it combines a true morphological analysis, phenotypic identification of the cells by fluorescent immunolabelling and detection of the presence of particular DNA sequences at the genomic level, such as rearrangements that are characteristic of cancer cells.

The combination, according to the method of the invention, of two different detection technique and of two different levels of analysis, applied on one and the same biological sample, and preferably a blood sample, gives this diagnostic technique increased sensitivity and reliability. More particularly, the method according to the present invention constitutes a diagnostic assay making it possible to give the indication of an antineoplastic treatment targeted against the oncogenic protein ALK in cancer patients.

The method of detection according to the invention is now described in detail.

The present invention relates to a method of identification, in a biological sample, in particular in a blood sample, of circulating tumour cells (CTCs) bearing at least one marker characteristic of the tumorous nature of the cell, said marker being selected from the groups constituted by:
the oncogenic proteins characteristic of the CTCs, and
the tumour markers.

said method comprising the following steps:
a. Identifying, on a support comprising the cells originating from the biological sample, at least one signal indicative of the presence of CTCs, in particular the signal characterizing the presence of a protein encoded by a gene that is characteristic of the tumorous nature of the cell,
b. Detecting, on a support comprising the cells originating from the biological sample, using a technique of the FISH type, the signal associated with the presence of a gene that is characteristic of the tumorous nature of the cell,
c. Comparing the location, on the support, of the signals obtained in steps a and b and identifying the CTCs.

The invention has the advantage of combining, on the same support, phenotypic labelling and analysis by a method of the FISH type, which preserve the integrity of the CTCs.

According to a first particular aspect, the invention relates to a method of identifying, in a biological sample originating from a patient, circulating tumour cells (CTCs) bearing the EML4-ALK fusion gene, said method comprising the following steps:
a. Identifying, on a support comprising the cells originating from the biological sample, at least one signal indicative of the presence of CTCs, in particular the signal characterizing the presence of a protein encoded by the EML4-ALK fusion gene,
b. Detecting, on a support comprising the cells originating from the biological sample, using a technique of the FISH type, the signal associated with the presence of the EML4-ALK fusion gene,
c. Comparing the location, on the support, of the signals obtained in steps a and b and identifying the CTCs.

Still according to this first aspect, the invention relates to a method of identifying, in a biological sample, in particular in a blood sample, circulating tumour cells (CTCs) bearing the EML4-ALK fusion gene, said method comprising the following steps:
a. Identifying, on a support comprising the cells originating from the biological sample, at least one signal indicative of the presence of CTCs, in particular of the signal characterizing the presence of a protein encoded by the EML4-ALK fusion gene,

b. Detecting, on a support comprising the cells originating from the biological sample, employing a technique of the FISH type using probes specific for rearrangement or amplification, the signal associated with the presence of the EML4-ALK fusion gene,
c. Comparing the location, on the support, of the signals obtained in steps a and b and identifying the CTCs.

According to this first aspect of the invention, the method detects the presence of the EML4-ALK fusion gene, by the FISH technique, and identifies the CTCs morphologically and phenotypically, and in particular the presence of the protein encoded by said fusion gene.

Detecting the EML4-ALK rearrangement relates exclusively to cancer patients with non-small-cell lung cancer (also called non-small-cell bronchial cancer (NSCBC)). The EML4-ALK fusion protein has a powerful oncogenic activity and a key role in carcinogenesis in a subpopulation of patients with NSCBC. This activity, which can be blocked by small molecules, in particular tyrosine kinase inhibitors targeting ALK, is an important molecular target in the treatment of these cancers. It is well known that the CTCs in these tumours have an important mesenchymal component and that the methods based on the detection of epithelial CTCs are ineffective for capturing the CTCs in lung cancer. Taking account of the very low yield in number of CTCs in these approaches, in a clinical context it is not possible to use approaches that capture only epithelial CTCs for diagnosis of the presence of rearrangement, monitoring of the response to ALK inhibitors or even for early detection of resistance.

The biological sample, and in particular the blood sample, originates from a cancer patient.

According to a particular embodiment of the method of identification, the circulating tumour cells (CTCs) are of epithelial and mesenchymal origin. Within the meaning of the present invention, the method of identifying the CTCs is also called the CTC enrichment method or CTC characterization method. The three expressions are equivalent and can be used interchangeably. The method of enrichment used according to the present invention is the ISET method.

According to a particular embodiment of the method of identification, the cells originating from the biological sample serving for identifying at least one signal indicative of the presence of CTCs and for detecting the signal associated with the presence of the EML4-ALK fusion gene, are isolated according to their size, which varies from 8 μm to at least 40 μm.

According to a second particular aspect, the invention relates to a method of identifying, in a biological sample originating from a patient, circulating tumour cells (CTCs) bearing at least one marker characteristic of the tumorous nature of the cell, said marker being selected from the groups constituted by:
the oncogenic proteins characteristic of the CTCs, with the exception of the EML4-ALK fusion gene, and
the tumour markers.

said method comprising the following steps:
a. Identifying, on a support comprising the cells originating from the biological sample, at least one signal indicative of the presence of CTCs, in particular the signal characterizing the presence of a protein encoded by a gene that is characteristic of the tumorous nature of the cell, with the exception of a protein encoded by the EML4-ALK fusion gene,
b. Detecting, on a support comprising the cells originating from the biological sample, using a technique of the FISH type, the signal associated with the presence of a gene that is characteristic of the tumorous nature of the cell, with the exception of the EML4-ALK fusion gene,
c. Comparing the location, on the support, of the signals obtained in steps a and b and identifying the CTCs.

The method of detecting the CTCs described in the publication of Charpentier et al. (2011) is a method of detecting CTCs comprising, on the one hand, the combined use of three fluorescent markers and, on the other hand, a morphocytological analysis carried out by an experienced cytopathologist. This document does not disclose a method of detecting CTCs combining fluorescent phenotypic markers and an automated analysis of the morphology of the cells; nor does it disclose a method combining phenotypic detection of the cells by fluorescent immunolabelling and detection of the presence of particular DNA sequences at the chromosomal level. Finally, this document does not disclose a method of detection combining automated detection of the morphology of the cells, phenotypic detection by fluorescent immunolabelling and molecular detection using techniques of the FISH (Fluorescent In Situ Hybridization) type.

According to another aspect, the method of identification according to the invention comprises the following steps:
a. Identifying, on a support comprising the cells originating from the biological sample, at least one signal indicative of the presence of CTCs, in particular the signal characterizing the presence of a protein encoded by a gene that is characteristic of the tumorous nature of the cell with the exception of the gene of the fusion protein EML4-ALK,
b. Detecting, on a support comprising the cells originating from the biological sample, using a technique of the FISH type, the signal associated with the presence of the EML4-ALK fusion gene,
c. Comparing the location, on the support, the signals obtained in steps a and b and identifying the CTCs.

Still according to this other aspect, the method of identification according to the invention comprises the following steps:
a. Identifying, on a support comprising the cells originating from the biological sample, at least one signal indicative of the presence of CTCs, in particular the signal characterizing the presence of a protein encoded by a gene that is characteristic of the tumorous nature of the cell with the exception of the gene of the fusion protein EML4-ALK,
b. Detecting, on a support comprising the cells originating from the biological sample, employing a technique of the FISH type using probes specific for rearrangement or amplification, the signal associated with the presence of the EML4-ALK fusion gene,
c. Comparing the location, on the support, of the signals obtained in steps a and b and identifying the CTCs.

According to this aspect, the method detects, on the one hand, the presence of the EML4-ALK fusion gene and, on the other hand, the presence of a protein other than a protein encoded by said fusion gene.

The combination of markers of different nature in steps a and b of the method has the particular advantage of offering better characterization of the cells, and therefore greater reliability of the diagnosis.

According to a particular aspect, the method according to the invention comprises a step of enriching the biological sample with CTCs, prior to or inherent in the steps of identifying the different signals, the factor of enrichment of the cells with CTCs being comprised from about 1/100 to about 1/100,000.

When the support comprising the cells originating from the biological sample is a filter, in particular an ISET filter (marketed by the company Rarecell or Screencell), filtration of the blood sample leads, in a manner inherent to filtration, to enrichment of the cells with CTCs. The approach using filters generally makes it possible to obtain more cells for establishing the diagnosis. Filters are much more difficult than any other support (lack of flatness, problems of background noise and pores). The pore size of these filters determines the size of the cells that can be retained on the filter, it being possible for said size of the cells to vary from 8 μm to at least 40 μm.

When the support comprising the cells originating from the biological sample is a slide, the enrichment step takes place during immunomagnetic separation, and precedes deposition on the slide.

This enrichment is of the order of from a factor of about 1/100 to a factor of about 1/100,000, depending on the method of enrichment used, and varies from patient to patient.

According to a more particular aspect, the invention relates to a method in which the cells originating from the patient's biological sample are deposited on a suitable support that can be analysed using an instrument of the fluorescence microscope or scanner type, and said support can be a filter or a slide.

The support used for depositing the biological sample can be analysed in a microscope, for example a fluorescence microscope, a scanner, or any other instrument for reading a support, whether reading is manual or automated. As stated above, the support can be a filter, a slide, or any other support suitable for depositing the sample and for reading the signals in a suitable instrument. The inventors have thus developed an automated strategy using a slide scanner (in particular the ARIOL® system from LEICA), which consists of sorting the cells recovered after filtration (or after enrichment by negative selection) into 2 fractions, one CD45(+) and the other CD45(−). The CD45(−) fraction containing the different CTC populations is then extensively analysed to identify the purely epithelial CTCs, the mesenchymal CTCs, hybrid (epithelial and mesenchymal) as well as other populations that would not express these markers. After FISH, the FISH signals (ALK/EML4 rearrangement) are relocated in these different populations. This therefore allows overall characterization of the CTCs phenotypically and genotypically.

In the method according to the invention, the cells originating from the patient's biological sample are collected during filtration, for depositing on a filter, or are deposited on a slide after immunomagnetic separation, or by any suitable method known to a person skilled in the art.

The method according to the invention is now described more particularly in detail regarding step a, i.e. identification of at least one signal indicative of the presence of CTCs, in particular of the signal characterizing the presence of a protein encoded by a gene that is characteristic of the tumorous nature.

According to a particular aspect, the invention relates to a method in which the step of identifying at least one signal indicative of the presence of CTCs is carried out by means of fluorescent immunolabelling using at least one marker selected from one of the groups constituted by:
i. a marker of the protein encoded by the EML4-ALK fusion gene,
ii. a nuclear membrane marker or a nuclear marker,
iii. a haematopoietic cell marker,
iv. a marker of proteins selected from the group constituted by: markers of proteins characteristic of the epithelial cells and markers of proteins characteristic of the mesenchymal cells.

More particularly, the invention relates to a method in which the step of identifying at least one signal indicative of the presence of CTCs is carried out by means of fluorescent immunolabelling with combined use of at least two markers, each being selected from one of the groups constituted by:
i. a marker of the protein encoded by the EML4-ALK fusion gene,
ii. a nuclear membrane marker or a nuclear marker,
iii. a haematopoietic cell marker,
iv. a marker of proteins selected from the group constituted by: markers of proteins characteristic of the epithelial cells and markers of proteins characteristic of the mesenchymal cells.

Even more particularly, the invention relates to a method in which the step of identifying at least one signal indicative of the presence of CTCs involves the combined use of at least three markers, each being selected from one of the groups constituted by:
i. a marker of the protein encoded by the EML4-ALK fusion gene,
ii. a nuclear membrane marker or a nuclear marker,
iii. a haematopoietic cell marker,
iv. a marker of proteins selected from the group constituted by: markers of proteins characteristic of the epithelial cells and markers of proteins characteristic of the mesenchymal cells.

According to an even more particular aspect, the invention relates to a method in which the step of identifying at least one signal indicative of the presence of CTCs involves the combined use of four markers, each being selected from one of the groups constituted by:
i. a marker of the protein encoded by the EML4-ALK fusion gene,
ii. a nuclear membrane marker or a nuclear marker,
iii. a haematopoietic cell marker
iv. a marker of proteins selected from the group constituted by: markers of proteins characteristic of the epithelial cells and markers of proteins characteristic of the mesenchymal cells.

In another aspect of the invention, regarding step a, the invention relates to a method in which the step of identifying at least one signal indicative of the presence of CTCs is carried out by means of fluorescent immunolabelling using at least one marker selected from one of the groups constituted by:
i. a marker of a protein encoded by a gene that is characteristic of the tumorous nature of the cell, with the exception of a marker of the protein encoded by the EML4-ALK fusion gene,
ii. a nuclear membrane marker or a nuclear marker,
iii. a haematopoietic cell,
iv. a marker of proteins selected from the group constituted by: the markers of proteins characteristic of the epithelial cells and the markers of proteins characteristic of the mesenchymal cells.

More particularly, the invention relates to a method in which the step of identifying at least one signal indicative of the presence of CTCs is carried out by means of fluorescent immunolabelling with combined use of at least two markers, each being selected from one of the groups constituted by:
i. a marker of a protein encoded by a gene that is characteristic of the tumorous nature of the cell, with the exception of a marker of the protein encoded by the EML4-ALK fusion gene,
ii. a nuclear membrane marker or a nuclear marker,
iii. a haematopoietic cell,
iv. a marker of proteins selected from the group constituted by: markers of proteins characteristic of the epithelial cells and markers of proteins characteristic of the mesenchymal cells.

Even more particularly, the invention relates to a method in which the step of identifying at least one signal indicative of the presence of CTCs involves the combined use of at least three markers, each being selected from one of the groups constituted by:
i. a marker of a protein encoded by a gene that is characteristic of the tumorous nature of the cell, with the exception of a marker of the protein encoded by the EML4-ALK fusion gene,
ii. a nuclear membrane marker or a nuclear marker,
iii. a haematopoietic cell marker,
iv. a marker of proteins selected from the group constituted by: markers of proteins characteristic of the epithelial cells and markers of proteins characteristic of the mesenchymal cells.

According to an even more particular aspect, the invention relates to a method in which the step of identifying at least one signal indicative of the presence of CTCs involves the combined use of four markers, each being selected from one of the groups constituted by:
i. a marker of a protein encoded by a gene that is characteristic of the tumorous nature of the cell, with the exception of a marker of the protein encoded by the EML4-ALK fusion gene,
ii. a nuclear membrane marker or a nuclear marker,
iii. a haematopoietic cell marker,
iv. a marker of proteins selected from the group constituted by: markers of proteins characteristic of the epithelial cells and markers of proteins characteristic of the mesenchymal cells.

According to another particular aspect, the invention relates to a method in which the marker specific for the protein encoded by the EML4-ALK fusion gene is the clone 5A4 or D5F3, marketed by the companies AbCam®, Cell Signaling Technology®.

In another particular aspect, the invention relates to a method in which the nuclear membrane marker used is the marker Emerin.

The step of morphological characterization is based either on the introduction of a nuclear membrane marker (such as emerin) in quadruple fluorescent labelling, or on carrying out a step of staining the nuclei (such as haematoxylin/eosin) after IF labelling, which must also be removed in the intermediate washing step between IF and FISH.

According to another particular aspect, the invention relates to a method in which the nuclear marker used is selected from the group constituted by: the marker DAPI, the markers Syto59, Sytox Orange, TOPRO 3, Hoechst 33342.

According to another particular aspect, the invention relates to a method in which the haematopoietic cell marker is selected from the group constituted by: CD45 and CD31. The marker used is preferably CD45.

Although very rare, the CTCs are known to be heterogeneous and composed of different subpopulations. The approach according to the invention makes it possible to identify and characterize the different subpopulations of CTCs, purely epithelial, purely mesenchymal, hybrid (epithelial and mesenchymal) as well as other populations that would not express these markers. Only the approaches of filtration or of negative selection of the CTCs by removing the haematopoietic cells can give access to these cells.

This is important from the standpoint of clinical application at two levels:
more CTCs (by number) are identified and it therefore becomes possible to use them for diagnosing the presence of ALK rearrangement and thus for deciding on the administration of a treatment with ALK inhibitors, then searching for the presence of CTCs having the ALK rearrangement during this treatment in order to assess its efficacy; the methods described in the prior art allow recovery of only a small fraction of the CTCs (epithelial CTCs) and this fraction is too rare and cannot be used in this clinical context. Furthermore studies show that the CTCs bearing an ALK rearrangement are mainly mesenchymal and thus cannot be or are very poorly characterized on the basis of methods of the prior art,
the emergence of a small minority of subpopulations bearing other molecular abnormalities, which will become the manifestation of a mechanism of resistance to treatments targeting ALK, become detectable; it is important to detect this resistance early in order to be able to change the treatment more quickly and allow the patient to benefit from a more appropriate treatment, the identification of the nature of the resistance is an important application of the CTCs, as there is no alternative means (impossibility of taking repeated biopsies) of early labelling and identification of the nature of this resistance which can be other molecular abnormalities (amplification of genes or mutations, etc.).

The methods according to the present invention can therefore genuinely contribute to improved therapeutic management of patients.

According to another particular aspect, the invention relates to a method in which the marker of proteins characteristic of the epithelial cells is selected from the group constituted by: the EpCAM markers, pan-cytokeratin markers and epithelial cadherin markers.

According to another particular aspect, the invention relates to a method in which the marker of proteins characteristic of the mesenchymal cells is selected from the group constituted by: the vimentin markers and neural cadherin markers.

A particular embodiment of the invention comprises a step of identifying at least one signal indicative of the presence of CTCs carried out by means of fluorescent immunolabelling using: i. the marker of the protein encoded by the EML4-ALK fusion gene,
i. the nuclear marker DAPI,
ii. the haematopoietic cell marker CD45,
iii. a pan-cytokeratin marker.

Another particular aspect of the invention relates to a method of the FISH type comprising the following successive steps:
enzymatic treatment of the support after binding,
hybridization on the support in the presence of at least one probe,
location of the hybridized probe on the support.

The method according to the invention comprises a FISH assay carried out according to a particular, advantageous protocol, developed by the inventors, and using probes specific for rearrangement or amplification, and not centromeric probes. This method therefore allows specific detection of the presence of genes rearranged or amplified in the cells.

When FISH assays are carried out on CTCs according to standard protocols of the prior art, the cells, which are very fragile, do not withstand the assays, and therefore the results of these assays are unusable.

Combining immunofluorescence (IF) and FISH is in fact very difficult for the following reasons:
the CTCs are by their nature very fragile owing to detachment from the tumour and it is generally assumed that a very high proportion of them are in apoptosis,
the sequence IF plus FISH requires methods that are very gentle both for IF and for FISH, with an intermediate step of removing the fluorescence before FISH. If the fluorescence is poorly removed, this results in a high level of background noise, making it impossible to interpret the FISH. Moreover, it is recognized that FISH is in itself already very difficult. The inventors have developed successive mild washings which allow for the fragility of the CTCs and avoid cellular losses,
in view of the rare nature of the CTCs, it is important to preserve the largest possible number of them. When working on filters, it is essential to heat the filters for 5 min at 98° C. to anchor the cells in the filter, otherwise the cells are lost.

According to a particular aspect, the method according to the invention is carried out on a patient's blood sample. The biological samples necessary for diagnosing the presence of a rearrangement of the ALK genes and for deciding on an anti-ALK targeted treatment in the assays according to the prior art are necessarily tumour biopsies, which are invasive and often of poor quality, in particular in patients with certain cancers such as NSCBC.

Another aspect relates to the use of a method according to the invention for analysing a biological sample originating from a cancer patient having the translocated ALK gene for monitoring tumour progression, for predicting a metastatic event or for measuring the efficacy of an anti-cancer treatment.

Another aspect relates to the use of a method according to the invention for analysing a biological sample originating from a cancer patient having the translocated ALK gene for diagnosing the indication of a tumour treatment against the ALK protein.

More particularly, the invention relates to the use of the method described in the context of patients with a cancer that is likely to lead to the presence of metastases, in particular non-small-cell bronchial cancer, and any other cancer having a rearrangement of the ALK gene.

A method according to the invention therefore comes under the category of personalized medicine with targeted antineoplastic therapy.

The invention is now described in more detail using examples and figures accompanying them.

### LEGENDS OF THE FIGURES

FIG. 1: Image from fluorescence microscopy on an ISET filter showing the labelling obtained on the H2228 line (bearing the EML4-ALK translocation) with the monoclonal antibody (5A4 or D5F3) specific to the translocated ALK protein. 1A. Labelling obtained with the nuclear stain DAPI; 1B. Labelling obtained with the antibody specific to the ALK protein; 1C. Labelling obtained with the antibody specific to the ALK protein and DAPI.

FIG. 2: Image from fluorescence microscopy showing a FISH experiment on an ISET filter carried out on the CTCs of a patient with non-small-cell bronchial carcinoma. 2A. Example of a haematopoietic cell retained on the filter that does not have a translocation; 2B and 2C. Example of two CTCs having a translocation. Probes specific to the 2p23 breakpoint were used.

FIG. 3: Images from fluorescence microscopy in three representative patients (P2, P6, P11) showing FISH detection of the ALK rearrangement in the CTCs and the tumour of the same patient. Different examples of CTCs are shown:: individual CTCs bearing only the ALK rearrangement (P2), individual CTCs bearing both a gain native ALK gene and the ALK rearrangement (P6), CTCs in clusters bearing the ALK rearrangement (P11).

FIG. 4: Microscopy images illustrating the method combining 4-colour immunofluorescence labelling (vimentin/cytokeratins/CD45/DAPI and N-cadherin/E-cadherin/CD45/DAPI) and FISH for detecting CTCs bearing the ALK rearrangement. 4A: Example of two patients showing the mesenchymal phenotype of the CTCs bearing the rearrangement ALK; 4B: Positive control of the immunofluorescence markers on the bronchial cancer line A549.

FIG. 5: Images from fluorescence microscopy illustrating the possibility of using FISH for monitoring the presence of CTCs bearing the ALK rearrangement in patients with ALK-positive NSCBC and treated with an ALK inhibitor. Example of CTCs detected in a patient before and on day 30 of treatment with crizotinib. Use of the method makes it possible to demonstrate in this patient the considerable decrease in the size and number of clusters of CTCs, some of which carry the ALK rearrangement.

FIG. 6: Image from fluorescence microscopy showing detection of HER2 amplification on an ISET filter. 6A. Example of a FISH experiment on the SKBR3 cell line; 6B. Example of a FISH experiment on the CTCs of a female patient who has the HER2 amplification; 6C: Example of immunofluorescence labelling with the monoclonal antibody directed against the HER2 protein on the CTCs of a patient who has the HER2 amplification.

FIG. 7: Image from fluorescence microscopy showing the use of FISH for detecting various molecular abnormalities in cell lines and patients with prostate cancer or breast cancer collected on an ISET filter. A: Detection of amplification of the ERG gene in the LnCAP cell line; B: Detection of the rearrangement of the ERG gene in the VCAP cell line; C: Detection of amplification of the AR gene in the LnCAP cell line; D: Detection of amplification of the HER2 gene in the SKBR3cell line; E: Detection of amplification of the ERG gene in a patient with prostate cancer; F: Detection of amplification of the cMYC gene in a patient with prostate cancer; G: Detection of amplification of the AR gene in a patient with prostate cancer; H: Detection of amplification of the HER2 gene in a patient with breast cancer.

FIG. 8: Microscopy images illustrating the automated method combining, on an ISET filter, 4-colour immunofluorescence labelling (vimentin/cytokeratins/CD45/DAPI) and FISH for detecting CTCs bearing amplification of the ERG or cMYC or AR genes, in cell lines (FIG. 8A) and CTCs of patients with prostate cancer (FIG. 8B).

FIG. 9: Microscopy images illustrating the method combining, on an ISET filter, immunofluorescence labelling (cMet/CD45/DAPI) and FISH in the H1975 bronchial cancer cell line; 9A 3-colour immunofluorescence detection of the cMET protein; 9B: Detection of cMET amplification; 9C: Method combining immunofluorescence labelling and FISH.

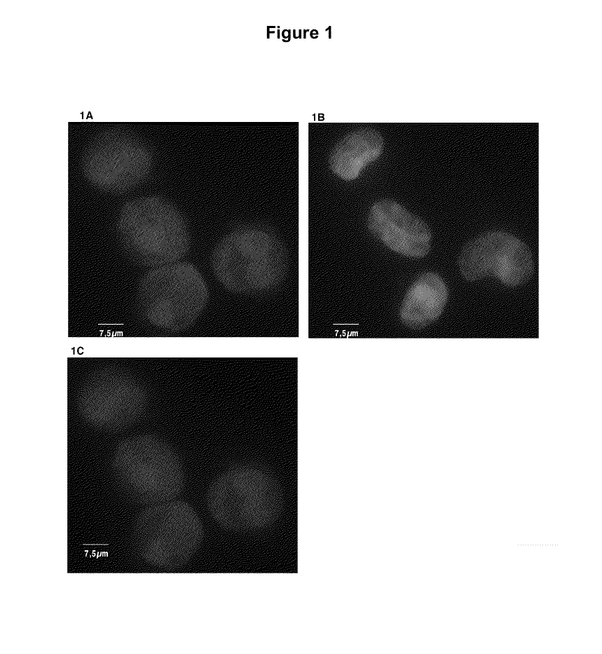

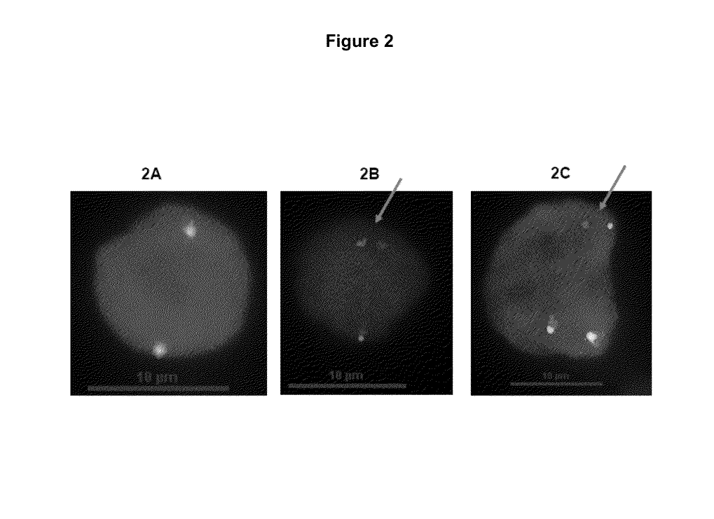

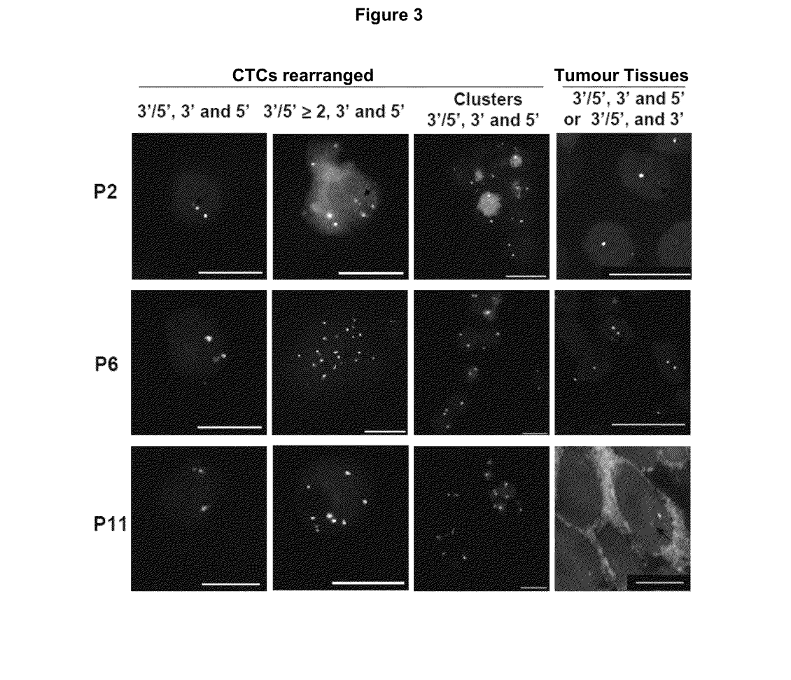

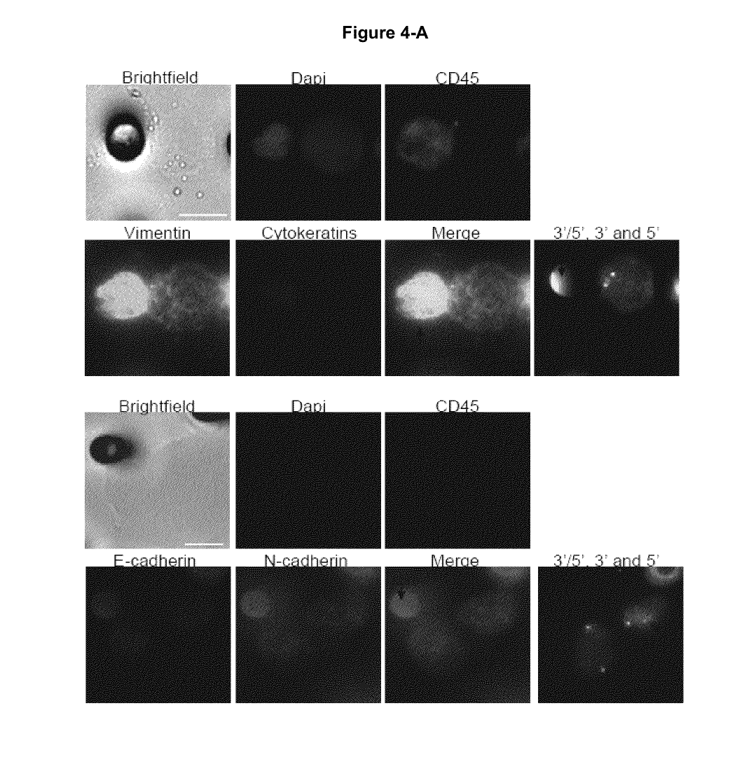

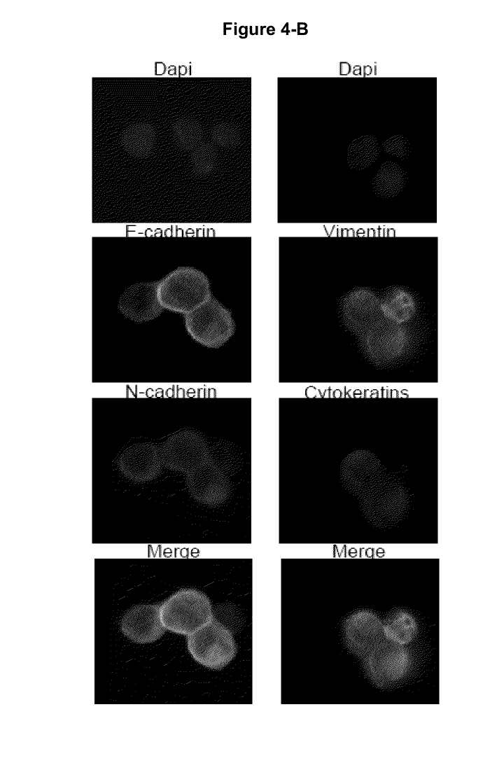

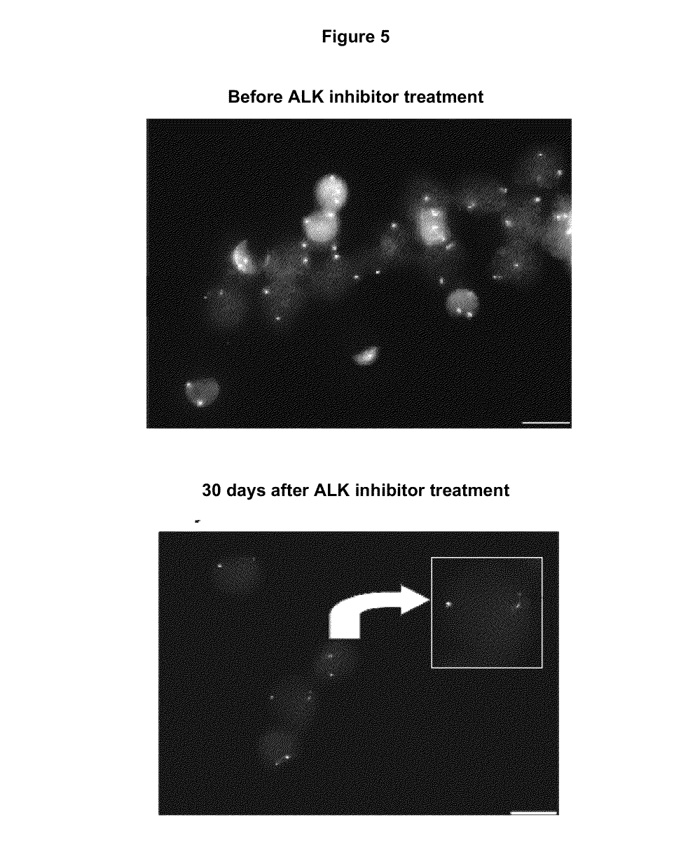

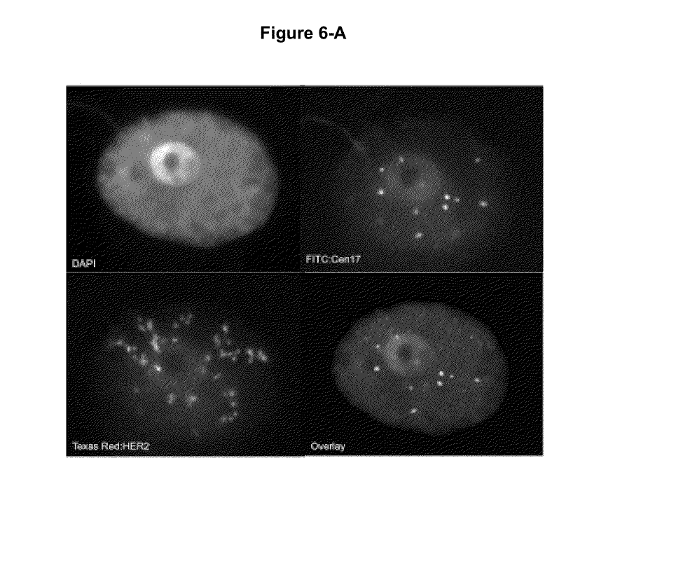

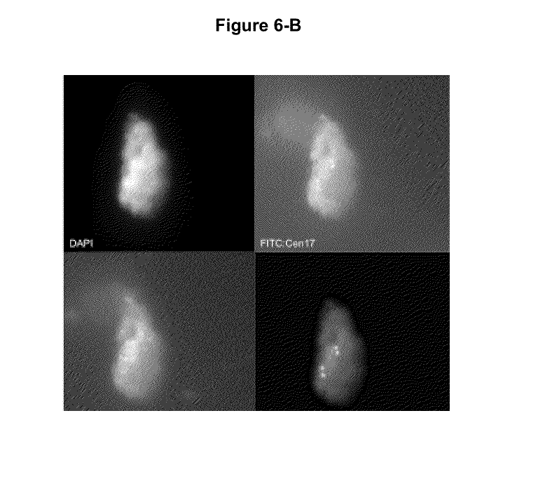

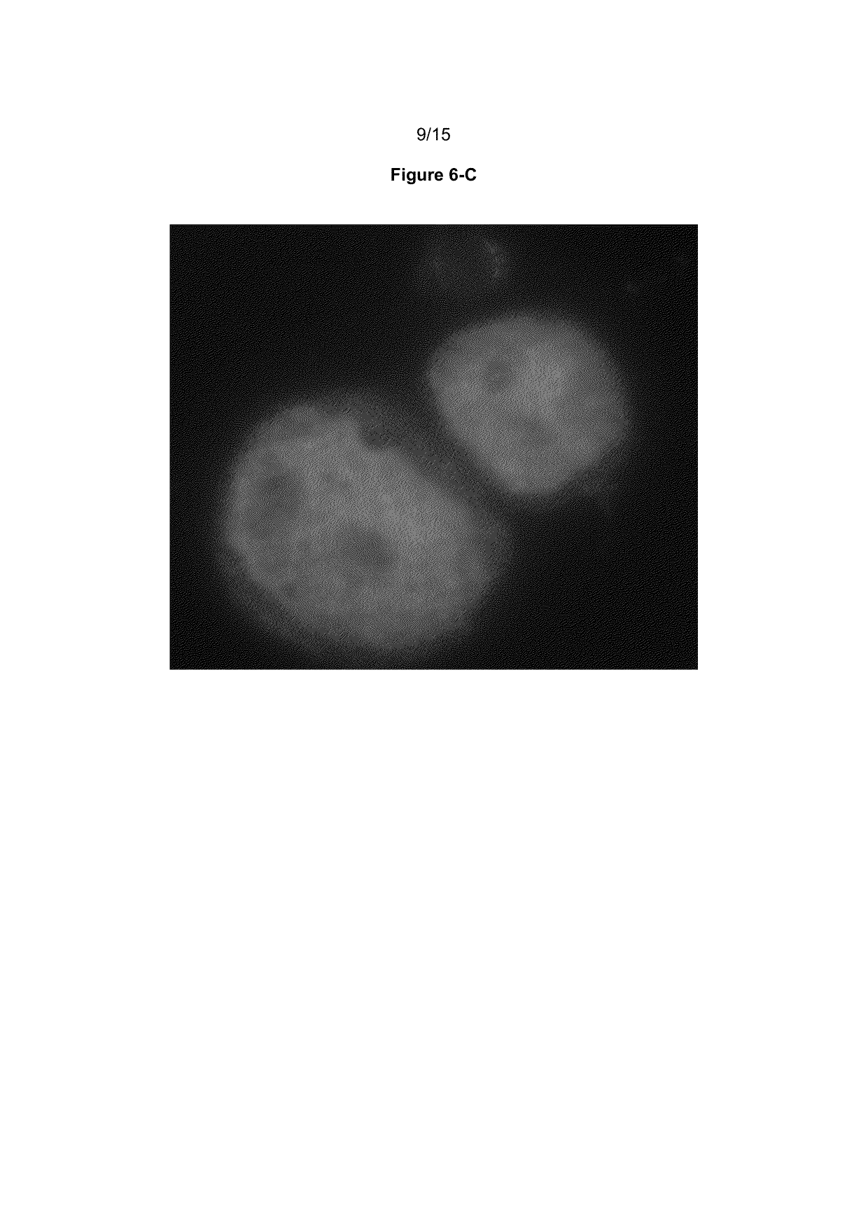

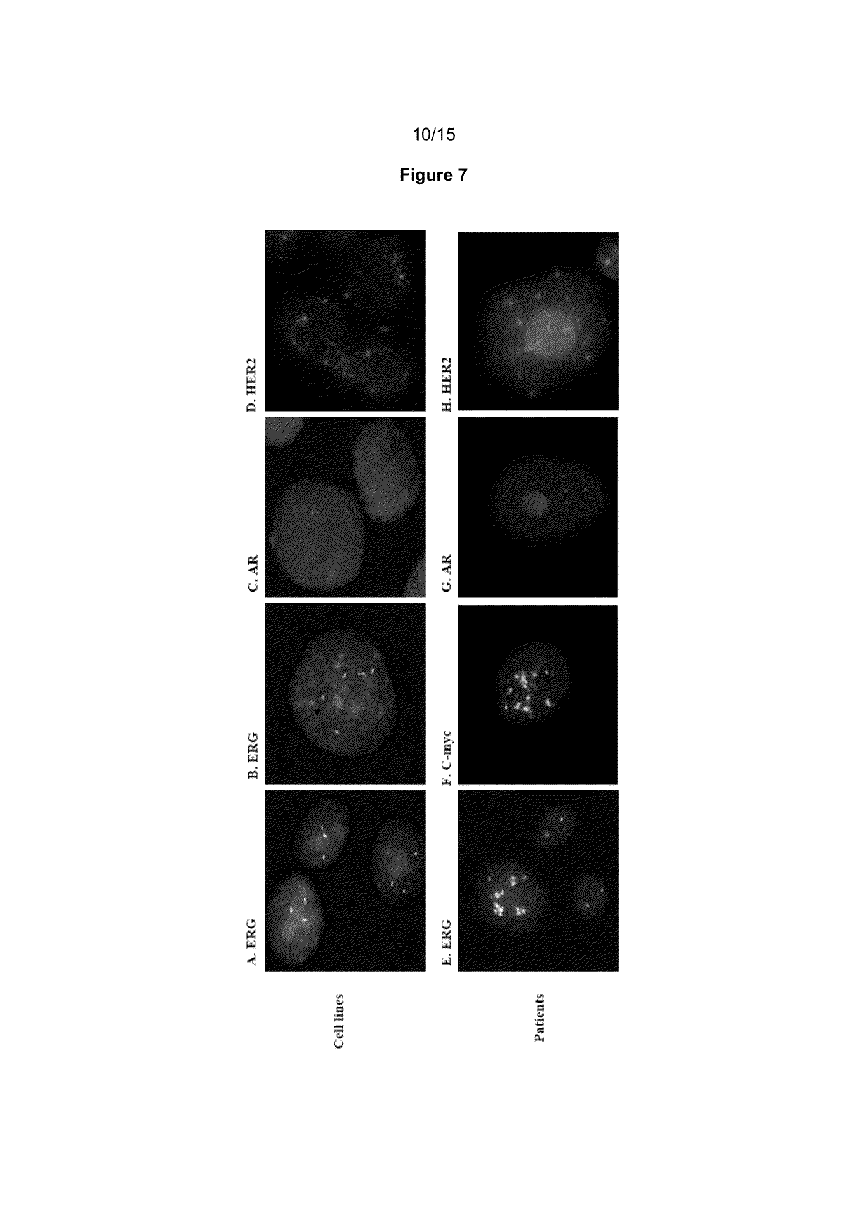

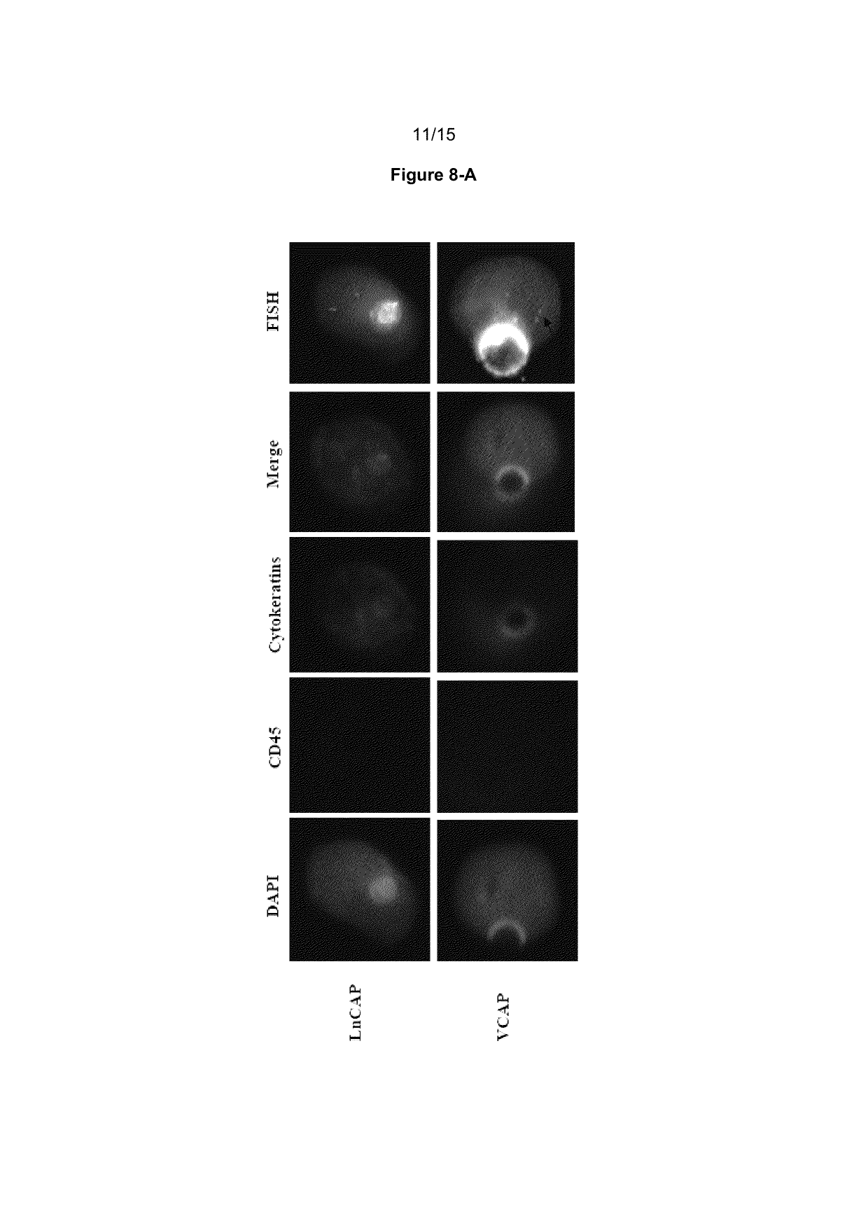

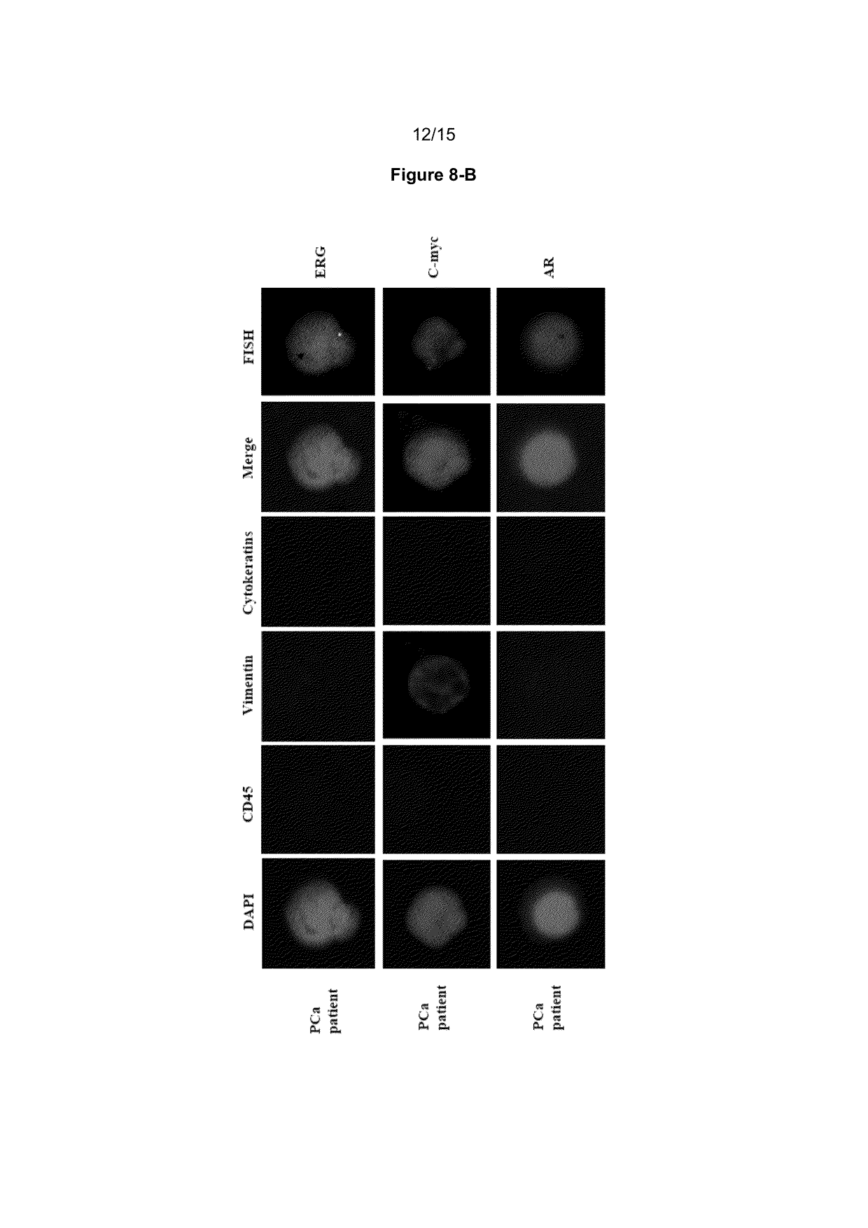

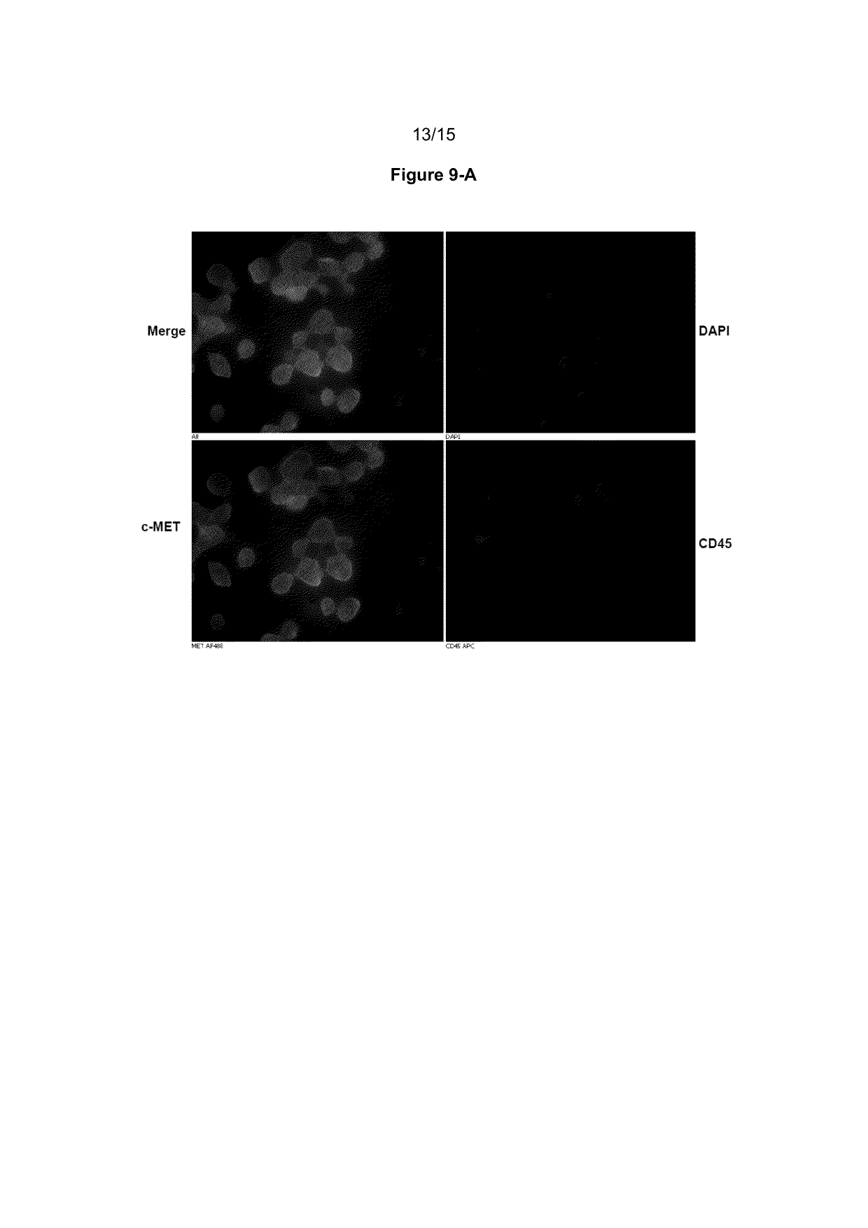

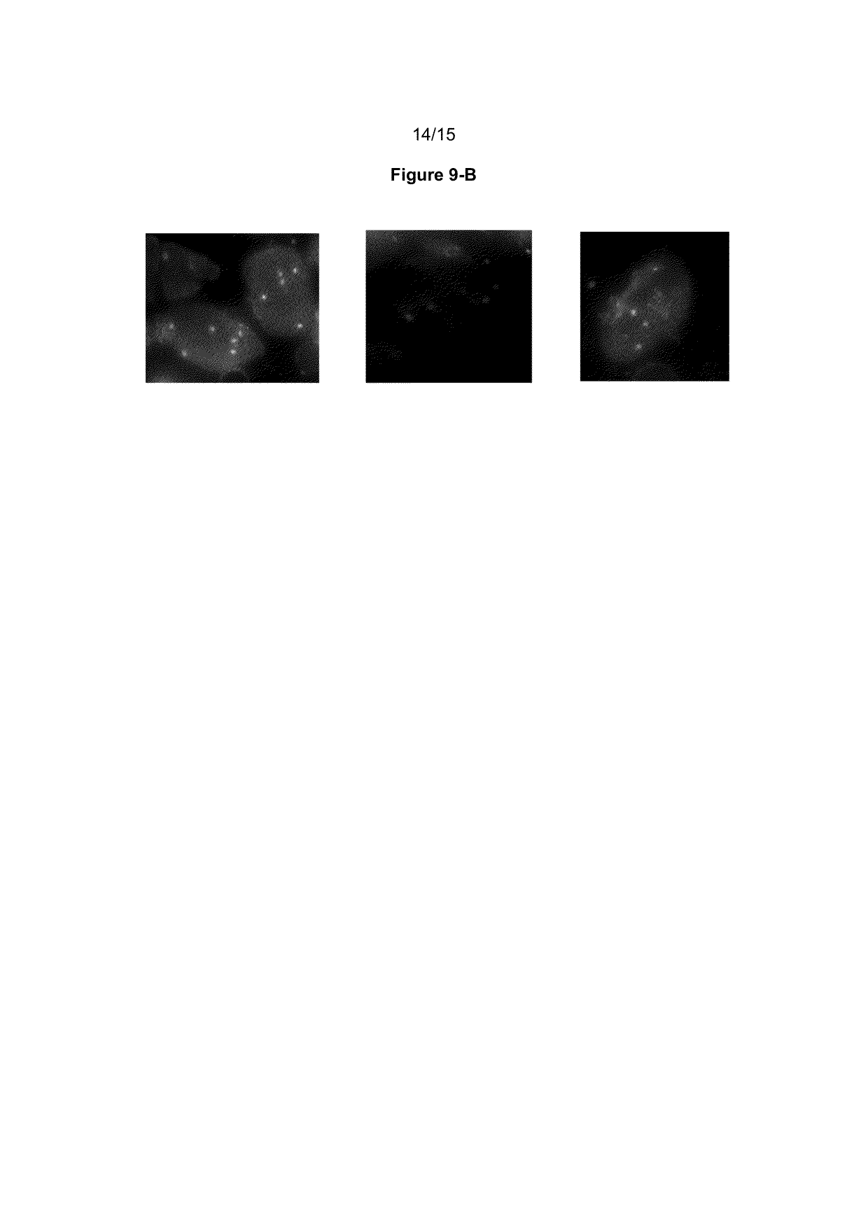

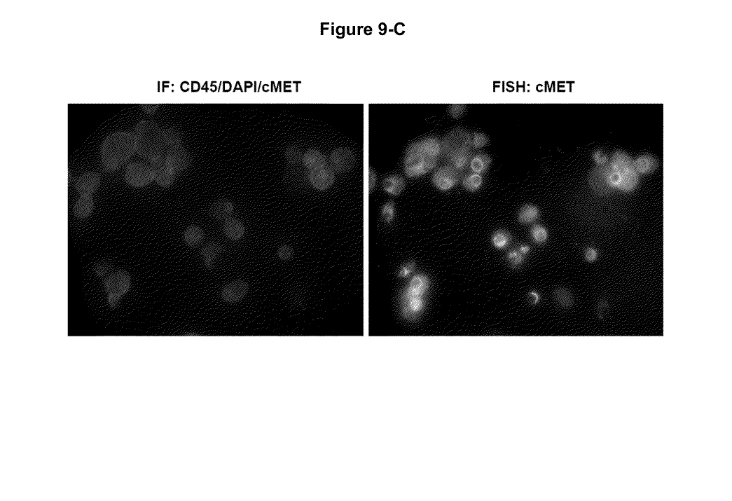

### EXAMPLES

### Example 1

### Preparation of the Analysis Support

The blood sample is enriched with CTCs by filtration based on the ISET technique (isolation by size of epithelial tumour cells). The filters are subjected to filtration at very low pressure on the ISET machine (7 mbar), and then dried on a heating stage at 45° C. for 2 min.

The filters are wrapped in aluminium foil and then frozen.

### Example 2

### Morphological and Phenotypic Identification of the CTS

The cells are identified by immunofluorescent labelling, on an ISET filter, of the nuclei, the cytokeratin, CD45 and the ALK protein.

The EDTA 1× pH9 buffer is prepared from EDTA 10×, and is then heated to 98° C.

Preparation of the Antibodies:

The antibodies are diluted in TBS-Triton at 0.4%, to a total volume of 100 μl. The different solutions of antibodies are prepared independently and then mixed.

The anti-cytokeratin antibodies (CK A1/A3—DAKO) are coupled to the AF546 fluorochrome according to the coupling protocol of the Zenon IGAF546 kit (Invitrogen).

30 μl of anti-CD45 APC antibody (BD Biosciences) is taken and will be added to the mixture of the different antibodies.

The anti-ALK antibodies (Novo-Castra) are coupled to the AF488 fluorochrome according to the coupling protocol of the Zenon IGAF488 kit (Invitrogen).

Prepared as follows: 4 μl of antibody to be coupled with the Zenon IgAF488 “mouse” coupling kit is added to 5 μl to IgAF488 and incubated for 5 min at ambient temperature. 5 μl of AF488 blocker is added and incubated for 5 min at ambient temperature. The antibody solution is stable for 30 min.

The different antibody solutions are combined and their volume is made up to 100 μl in TBS-Triton 0.4%.

Filter Preparation:

The filters are removed from the freezer and placed at ambient temperature for approximately 15 min. The filters are fixed on the slide using adhesive tape that is resistant to high temperatures, and the latter also makes it possible to identify the filters. The filters are then rehydrated in TBS for 5 min and then drained.

Unmasking and Immunolabelling:

The slides are immersed for 5 min in the EDTA 1× pH 9 buffer heated on a water bath to 98° C., then they are rinsed for 2 min in TBS and for 1 min with distilled water and then drained. On the filter, the spots (8 mm diameter) are circled using a DakoPen (hydrophobic). Humid chambers are prepared using absorbent paper previously passed under tap water. 100 μl of the prepared antibody solution is deposited on each spot, then each spot is covered with a round slip with diameter of 12 mm. The whole is incubated overnight in a humid chamber at 4° C. in the dark.

After incubation, the chambers are kept at ambient temperature for 15 min and then rinsed in TBS-Tween at 0.05% for 2 min and drained, rinsed in distilled water and then drained.

100 μl of previously diluted DAPI mounting solution is deposited on each slide, the whole is incubated for 15 min at ambient temperature in the dark.

The slides are then rinsed in TBS-Tween at 0.05% for 2 min and then drained, then rinsed in distilled water and drained.

The slides and cover slips are then mounted in the fluoromount; after 1 h at ambient temperature in the dark, they are sealed, before being stored at 4° C.

Reading is carried out with the microscope on the next day; the CTCs are identified, and their positions are recorded.

### Example 3

### Assay for Detecting the EML4-ALK Fusion Gene by the FISH Technique

his assay is carried out after immunofluorescence labelling.

### A. Fixing of the Material

After analysis of the immunofluorescence results, the slide and cover slip system is dismantled and the filter is washed in a solution of PBS 1× before being fixed with a solution of methanol/acetic acid (9:1) for 2 hours at ambient temperature.

### B. Treatment of the Filter Immobilized on the Slide

Preparation of the 10% Pepsin:

Weigh 1 g to be dissolved in 10 ml H₂O, under magnetic stirring, in a conical flask. Once well dissolved, put the conical flask in ice. Take 100 μl aliquots and store them at −20° C.

A solution of 80 ml of HCl at 0.01 N (8 ml HCl 0.1 +72 ml H2O) is preheated to 37° C. in a water bath.

The slides stored at +4° C. are incubated for 5 min in a bath of 4×SSCT.

An aliquot of pepsin is added to the 0.01 N HCl, at 37° C. The filter is incubated for several minutes in the pepsin solution, stirring every two minutes.

A fixing solution, formaldehyde with a final concentration of 1%, is prepared extemporaneously.

The solution obtained is stable for one day.

The slides are washed in a container of PBS1× for 5 min without wetting the adhesive tape and without stirring. The slides are then fixed by immersing in the fixing solution for 2 min, without wetting the adhesive tape. The slides are then dehydrated for two minutes with 70%, 85% and 100% ethanol successively, without leaving the slides to dry between the alcohols. The back of the slide is wiped carefully, the filter is then dried for several minutes, and for one hour at most, on a plate at 37° C., or it is dried at ambient temperature.

### C. Denaturation of the Slide and Hybridization (D1)

In darkness, a sealed box is placed on the illuminated stage at 37° C.; this box will allow the slide to be transported if required. The apparatus for hybridization, or “hybridizer”, is switched on and programmed to reach a temperature of 37° C. The slides are inserted into their respective places, in the hybridizer. The two strips on the cap are moistened with water, then replaced on the cover of the hybridizer to minimize evaporation of the probe. The slide is placed on the stage at 37° C. 10 μl of ALK probe hybridization mixture (Vysis) is deposited on each spot (8 mm diameter) (7 μl of buffer, 2 μl of water, 1 μl of probe are deposited for each spot). A small round slip (12 mm diameter) is placed carefully on the spots. The zone of interest is sealed with glue placed all round, in order to isolate this zone completely. The glue is dried by placing the slide on the stage at 37° C. for about 5 min. The slide is then placed in the hybridizer immediately so as not to alter the temperature of the probe.

The denaturation programme is started at 85° C. for 10 min, then hybridization takes place conventionally at 44° C. overnight (12 hours) at 44° C. This hybridization can be carried out in the hybridizer or in a stove set at 44° C. In the latter case, after the step at 85° C., the slide is very quickly placed in the box already at 37° C. on the stage, then transferred into the special humid black box placed in the stove at 44° C. in advance. The hybridizer is stopped when it has cooled to 37° C.

### D. Washing and Mounting (D2)

The water bath is switched on at 65° C. and the plate at 37° C.; Wash Buffer 1× Dako is prepared extemporaneously.

The buffer is then preheated, putting half of the buffer in a porcelain container that is placed in the water bath at 65° C. before the temperature is reached (to avoid thermal shocks); the temperature is reached after about 30 min.

The other half of the buffer is put in a container for slides, at ambient temperature. The slide is taken out of the hybridizer or stove and transported if necessary in a special black box to protect the slide from the light.

Without light, the glue is removed very gently with tweezers, without damaging the filter. The slide is washed for approximately 7 min in Stringent Wash Buffer at ambient temperature without stirring. The slide is washed for 5 min in the buffer preheated to 65° C., and then for 5 min in Wash Buffer 1× DAKO at ambient temperature.

The slide is dehydrated for 2 min with 70%, 85% and 100% ethanol successively, without being left to dry between the alcohols.

The back of the slide is wiped carefully with absorbent paper, and is then left to dry for several minutes on the plate at 37° C., checking that there is no longer any alcohol between the slide and the filter.

If there is background noise, the slides can be washed again at 70° C. for 1-5 min.

From 15 to 20 μl of “antifFad+DAPI” solution is deposited on the filter and distributed as several drops on the surface of the filter.

The slides are covered with a 22×22 or 24×40 cover slip, the whole is sealed on one side of the cover slip, on the side opposite the spot.

The slide is dried on the heating stage at 37° C. for several minutes approximately.

The slide is transported in a black box to the microscope, and observed using ×20 magnifications successively to locate the spot, and then ×100, with immersion oil.

The slide can be stored in the dark for 1 week at ambient temperature or for 1 year at −20° C.

Reading is carried out with the fluorescence microscope or scanner. The CTCs are located again from the positions already identified during the immunolabelling.

### Example 4

### Assay for Detecting the HER-2 Gene by the FISH Technique

This assay is carried out after immunofluorescence labelling.

### A. Fixing of the Material

After analysis of the immunofluorescence results, the slide and cover slip system is dismantled and the filter is washed in a solution of PBS 1× before being fixed with a solution of methanol/acetic acid (9:1) for 2 hours at ambient temperature.

### B. Treatment of the Filter Immobilized on the Slide

Preparation of 10% Pepsin:

Weigh 1 g to be dissolved in 10 ml H₂O, under magnetic stirring, in a conical flask. Once well dissolved, put the conical flask in ice. Take 100 μl aliquots and store them at −20° C.

A solution of 80 ml of HCl at 0.01 N (8 ml HCl 0.1 N+72 ml H2O) is preheated in a water bath at 37° C. for at least 30 min.

The slides stored at +4° C. are incubated successively for 5 min in a bath of 4×SSCT to detach the cover slip; if necessary the cover slip can be detached using tweezers, the mounting medium is removed and then the filter is rinsed in a bath of PBS 1×.

An aliquot of pepsin is added to the 0.01 N HCl, at 37° C., and the whole is mixed thoroughly. The filter is incubated for 6 min in the pepsin solution, stirring every two minutes.

A fixing solution containing 1% formaldehyde is prepared extemporaneously.

The slides are washed in a container of PBS1× for 5 min without wetting the adhesive tape and without stirring. The slides are then fixed by immersing in the fixing solution for 2 min, without wetting the adhesive tape. The slides are then dehydrated for two minutes with 70%, 85% and 100% ethanol successively, without leaving the slides to dry between the alcohols. The back of the slide is wiped carefully, the filter is then dried for several minutes, and for one hour at most, on a plate at 37° C., or it is dried at ambient temperature.

### C. Denaturation of the Slide and Hybridization (D1)

In the dark, a sealed box is placed on the illuminated stage at 37° C.; this box will allow the slide to be transported if necessary.

The apparatus for hybridization, or “hybridizer”, is switched on and programmed to reach a temperature of 37° C. The slides are inserted into their respective places in the hybridizer. The two strips on the cap are moistened with water, then replaced on the cover of the hybridizer to minimize evaporation of the probe. The slide is placed on the stage at 37° C. 6 μl of HER-2 probe (Dako) is deposited on each spot (8 mm diameter).

A small round slip (12 mm diameter) is placed carefully on the spots. Any bubbles are carefully pushed outside of the surface of the cover slip, for example with a cone. The zone of interest is sealed with glue placed all round, in order to isolate this zone completely. The glue is dried by leaving the slide on the stage at 37° C. for about 5 min. The slide is then placed in the hybridizer immediately so as not to alter the temperature of the probe.

The denaturation programme is started at 85° C. for 10 min, then hybridization takes place at 44° C. overnight (12 hours) at 44° C. This hybridization can be carried out in the hybridizer or in a stove set at 44° C.

In the latter case, after the step at 85° C., the slide is placed very quickly in the box already at 37° C. on the stage, then transferred into the special humid black box placed in the stove at 44° C. in advance.

The hybridizer is stopped when it has cooled to 37° C.

### D. Washing and Mounting (D2)

The water bath is switched on at 65° C. and the plate at 37° C.; Wash Buffer 1× Dako is prepared extemporaneously.

The buffer is then preheated, placing half of the buffer in a porcelain container that is placed in the 65° C. water bath before the temperature is reached (to avoid thermal shocks); the temperature is reached after about 30 min.

The other half of the buffer is put in a container for slides, at ambient temperature. The slide is taken out of the hybridizer or stove and transported if necessary in a special black box to protect the slide from the light.

Without light, the glue is removed very gently with tweezers, without damaging the filter. The slide is washed for approximately 7 min in the buffer

Stringent Wash Buffer at ambient temperature without stirring. The slide is washed for 5 min in the buffer preheated to 65° C., and then for 5 min in Wash Buffer 1× DAKO at ambient temperature.

The slide is dehydrated for 2 min with 70%, 85% and 100% ethanol successively, without being left to dry between the alcohols.

The back of the slide is wiped carefully with absorbent paper, and is then left to dry for several minutes on the plate at 37° C., checking that there is no longer any alcohol between the slide and the filter.

If there is background noise, the slides can be washed again at 70° C. for 1-5 min.

Depending on the area of the cover slip, 10 to 15 μL of “antifFad+DAPI” solution is deposited on the filter and distributed in several drops over the surface of the filter.

The slides are covered with a 22×22 or 24×40 cover slip, the whole is sealed on one side of the cover slip. The slide is dried on a heating stage at 37° C. for about 5 min.

The slide is transported in a black box to the microscope, and observed using ×20 magnifications successively to locate the spot, and then ×100, with immersion oil.

The slide can be stored in the dark for 1 week at ambient temperature or for 1 year at −20° C.

Reading is carried out with the fluorescence microscope or scanner. The CTCs are located again from the positions already identified during the immunolabelling.

### Example 5

### Assay for Detecting the c-TEM Gene by the FISH Technique

The FISH technique is implemented in the same way as described in Example 4, using a probe specific for c-TEM.

### Example 6

### Assay for Detecting the ERG Fusion Gene by the FISH Technique

The FISH technique is implemented in the same way as described in Example 4, using a probe specific for Erg.

### REFERENCES

A. Lecharpentier et al., “Detection of circulating tumour cells with a hybrid (epithelial/mesenchymal) phenotype in patients with metastatic non-small-cell lung cancer” British Journal of Cancer, 2011, 1-4.

1. Method of identifying, in a biological sample, in particular in a blood sample, circulating tumour cells (CTCs) bearing the EML4-ALK fusion gene, said method comprising the following steps:
a. Identifying, on a support comprising the cells originating from the biological sample, at least one signal indicative of the presence of CTCs, in particular of the signal characterizing the presence of a protein encoded by the EML4-ALK fusion gene,
b. Detecting, on a support comprising the cells originating from the biological sample, employing a technique of the FISH type using probes specific for rearrangement or amplification, the signal associated with the presence of the EML4-ALK fusion gene,
c. Comparing the location, on the support, of the signals obtained in steps a and b and identifying the CTCs.

2. Method of identification, according to claim 1, in which the circulating tumour cells (CTCs) are of epithelial and mesenchymal origin.

3. Method of identification, according to claim 1, in which the cells originating from the biological sample serving for identifying at least one signal indicative of the presence of CTCs and for detecting the signal associated with the presence of the EML4-ALK fusion gene, are isolated according to their size, which varies from 8 μm to at least 40 μm.

4. Method of identification, according to claim 1, said method comprising the following steps:
a. Identifying, on a support comprising the cells originating from the biological sample, at least one signal indicative of the presence of CTCs, in particular the signal characterizing the presence of a tumour marker with the exception of the protein encoded by the fusion gene EML4-ALK,
b. Detecting, on a support comprising the cells originating from the biological sample, employing a technique of the FISH type using probes specific for rearrangement or amplification, the signal associated with the presence of the fusion gene EML4-ALK,
c. Comparing the location, on the support, of the signals obtained in steps a and b and identifying the CTCs.

5. Method according to claim 1, comprising a step of enriching the biological sample with CTCs, prior to or inherent in the steps of identifying the different signals, the factor of enrichment of the cells with CTCs being comprised from about 1/100 to about 1/100,000.

6. Method according to claim 1, in which the cells originating from the patient's biological sample are deposited on a suitable support that can be analysed using an instrument of the fluorescence microscope or scanner type, and said support can be a filter or a slide.

7. Method according to claim 1, in which the cells are collected on the support by filtration or deposited on the support after immunomagnetic separation.

8. Method according to claim 1, in which the step of identifying at least one signal indicative of the presence of CTCs is carried out by means of fluorescent immunolabelling using at least one marker selected from one of the groups constituted by:
i. a marker of the protein encoded by the EML4-ALK fusion gene,
ii. a nuclear membrane marker or a nuclear marker,
iii. a haematopoietic cell marker,
iv. a marker of proteins selected from the group constituted by: the markers of proteins characteristic of the epithelial cells and the markers of proteins characteristic of the mesenchymal cells.

9. Method according to claim 8, in which the step of identifying at least one signal indicative of the presence of CTCs is carried out by means of fluorescent immunolabelling with combined use of at least two markers, each being selected from one of the groups constituted by:
i. a marker of the protein encoded by the EML4-ALK fusion gene,
ii. a nuclear membrane marker or a nuclear marker,
iii. a haematopoietic cell marker,
iv. a marker of proteins selected from the group constituted by: the markers of proteins characteristic of the epithelial cells and the markers of proteins characteristic of the mesenchymal cells.

10. Method according to claim 8, in which the step of identifying at least one signal indicative of the presence of CTCs involves the combined use of at least three markers, each being selected from one of the groups constituted by:
i. a marker of the protein encoded by the EML4-ALK fusion gene,
ii. a nuclear membrane marker or a nuclear marker,
iii. a haematopoietic cell marker,
iv. a marker of proteins selected from the group constituted by: the markers of proteins characteristic of the epithelial cells and the markers of proteins characteristic of the mesenchymal cells.

11. Method according to claim 8, in which the step of identifying at least one signal indicative of the presence of CTCs involves the combined use of four markers, each being selected from one of the groups constituted by:
i. a marker of the protein encoded by the EML4-ALK fusion gene,
ii. a nuclear membrane marker or a nuclear marker,
iii. a haematopoietic cell marker,
iv. a marker of proteins selected from the group constituted by: the markers of proteins characteristic of the epithelial cells and the markers of proteins characteristic of the mesenchymal cells.

12. Method according to claim 1, in which the marker specific to the protein encoded by the fusion gene EML4-ALK is the clone 5A4 or D5F3.

13. Method according to claim 8, in which the nuclear membrane marker used is the marker Emerin.

14. Method according to claim 8, in which the nuclear marker used is selected from the group constituted by: the marker DAPI, the markers Syto59, Sytox Orange, TOPRO 3, Hoechst 33342.

15. Method according to claim 8, in which the haematopoietic cell marker is selected from the group constituted by: CD45 and CD31.

16. Method according to claim 8, in which the marker of proteins characteristic of the epithelial cells is selected from the group constituted by: the EpCAM markers, the pan-cytokeratin markers, the epithelial cadherin markers.

17. Method according to claim 8, in which the marker of proteins characteristic of the mesenchymal cells is selected from the group constituted by: the vimentin markers and the neural cadherin markers.

18. Method according to claim 8, in which the step of identifying at least one signal indicative of the presence of CTCs is carried out by means of fluorescent immunolabelling using:
i. the marker of the protein encoded by the EML4-ALK fusion gene,
ii. the nuclear marker DAPI,
iii. the haematopoietic cell marker CD45,
iv. a pan-cytokeratin marker.

19. Method according to claim 1, in which the method of the FISH type comprises the following successive steps:
enzymatic treatment of the support after binding,
hybridization on the support in the presence of at least one probe,
location of the hybridized probe on the support.

20. Method according to claim 1, in which the biological sample originating from a patient is blood.

21. A method for analysing a biological sample originating from a cancer patient having a translocated ALK gene, for predicting a metastatic event, comprising;
obtaining the biological sample; and
performing the method of claim 1 on the biological sample.

22. The method according to claim 21, in which the patient has a cancer that may lead to the presence of metastases, in particular non-small-cell lung cancer, prostate cancer, breast cancer.
